(19) United States
(12) Patent Application Publication
(10) Pub. No.: US 2011/0104698 A1
(43) Pub. Date: May 5, 2011

(54) MARKERS FOR DETERMINING DNA DAMAGE AND TELOMERE DYSFUNCTION FOR THE DETERMINATION OF THE BIOLOGICAL AGE, REGENERATIVE CAPACITY, CANCER RISK, THE RISK OF DEVELOPING AGE-RELATED DISEASES AND THE PROGNOSIS OF CHRONIC DISEASES IN HUMANS AND ANIMALS

(76) Inventor: Lenhard Rudolph, Ulm (DE)

(21) Appl. No.: 12/989,545

(22) PCT Filed: Apr. 27, 2009
(86) PCT No.: PCT/EP2009/055059

§ 371 Date: Jan. 13, 2011

(30) Foreign Application Priority Data
Apr. 25, 2008 (EP) 08155176.4

(51) Int. Cl. C12Q 1/68; G01N 33/573; G01N 33/68; G01N 33/53

(52) U.S. Cl. 435/6; 435/7.4; 436/86; 435/7.1

(57) ABSTRACT

Process for determining the presence and extent of DNA damage and telomere dysfunction in humans or animals, comprising the following steps:

determining the level or activity of at least one protein marker in a blood or serum sample, said protein being selected from the group consisting of EF1α, chitobiosidases, stathmin and CRAMP.

The present invention relates to markers that may be used for determining the biological ageing, regenerative capacity and prognosis in age-related and chronic diseases, especially markers that can be determined from blood or serum.

The number of elderly and chronically ill people is increasing in most countries of the world (1). The determination of biological ageing, the regenerative capacity or prognosis in chronic and age-related diseases is a fundamental medical problem. Biomarkers that could be used for such issues are not currently available. The identification of readily determined biomarkers that indicate the biological ageing, the regeneration capacity and the risk of disease in old age could be used for improving and individualizing therapies (start of therapy, therapy selection etc.) in old age and in chronic diseases. In addition, such markers can be used to develop medicaments, substances, food products/additives and regimens that can delay biological ageing.

In many age-related and chronic diseases, early diagnosis is of clinical importance. Early diagnosis and prognosis are clinically necessary to initiate a therapy that is exactly adapted to the specific disease and its individual course. This can reduce the risk that the patients may develop further consecutive diseases or complications. In addition, invasive therapies often also represent a high risk in elderly people. The estimation of the risk of side effects or complications of the therapy could lead to a better therapy selection. This appears to be indicated, in particular, for therapies requiring some regenerative reserve of the patient, such as surgery, chemotherapy or radiotherapy.

One of the few biological markers associated with ageing, age-related diseases and chronic diseases is the shortening of the telomeres. Telomeres form the end regions of chromosomes (2). In human cells, a shortening of the telomeres occurs in each cell division (3). This limits the proliferation capacity of human cells to 50-70 divisions (3). In humans, a shortening of the telomeres within the scope of ageing occurs in almost all tissues (4). The shortening of the telomeres correlates with the survival rate of 60-75 year old people (5). An accelerated shortening of the telomeres has been associated with age-related diseases such as Alzheimer's (6), diabetes mellitus (7), cardiovascular diseases (8) and tumor development (9). In addition, the shortening of the telomeres correlates with the progression of the disease and organ failure in chromic diseases such as hepatitis (10) and myelodysplastic syndromes (11).

The determination of the telomere length has not yet become established in hospitals, because technically complicated methods, such as Southern blotting, quantitative fluorescence, in situ hybridization or quantitative PCR, must be employed for this purpose. In addition, samples are often difficult to obtain. Thus, the telomere shortening in liver tissue correlates with the progress of chronic liver diseases towards liver cirrhosis (10). Therefore, it would be necessary to perform liver biopsies to be able to estimate the prognosis and the course of the disease.

Another problem of the determination of telomere length is the fact that the telomere length as such has limited significance to cell function and regenerative capacity. Animal tests have shown that the critical parameter is the number of critically short dysfunctional telomeres rather than the average telomere length (12). Thus, in a mouse model, premature ageing and reduction of organ preservation take place if the number of dysfunctional telomeres is increased, even though the average telomere length may still be relatively long (12). These results are also of importance to the proliferation capacity of human cells. Thus, the induction of senescence and thus an irreversible loss of proliferation of the cells occur if the number of dysfunctional telomeres per cell exceeds a certain extent (13).

To conclude, telomere dysfunction seems to be an indication of ageing, age-related diseases and chronic diseases. However, the determination of telomere dysfunction as a clinical marker has not become established since telomere dysfunction is difficult to determine in terms of methodology, and biopsies from the affected organs are often not available.

It is an object of the present invention to provide a process by which a determination of the biological ageing, regenerative capacity and prognosis in age-related and chronic diseases can be effected in a simple way.

This object is now achieved by the identification of marker proteins secreted by cells in response to telomere dysfunction (or other forms of DNA damage) that can be determined in the blood serum with simple methods.

A group of four proteins secreted by cells in response to telomere dysfunction or DNA damage have been identified Jiang, Rudolph, Schiffer, Mischak et al., 2008, and unpublished data). These proteins have been identified in the culture supernatant of bone marrow cells from telomerase knockout (Terc^{-/-}) mice with dysfunctional telomeres. In preliminary studies, it has been shown that Terc^{-/-} mice develop telomere dysfunction in bone marrow cells and that the function of hematopoietic stem and progenitor cells is limited thereby. For the identification of marker proteins of telomere dysfunction, bone marrow cells from these mice were subjected to short culturing (4 hours). Then, a proteoma analysis of the secreted proteins in the cell culture supernatant was performed by means of CE/TOF-MS (capillary electrophoresis/time-of-flight mass spectrometry). In this method, four proteins were identified that are specifically associated with the ageing of telomere-dysfunctional mice.

These proteins are:
1. Elongation factor 1-alpha (EF-1alpha): This protein controls the translational protein synthesis and is up-regulated in human cells in response to proliferation loss (senescence) (18, 19).
2. Chitinase-3-like protein 3 (Chi3L3): This protein belongs to the family of chitinases, which are also activated in response to activation of the innate immune system (15, 16). The up-regulation of a member of the chitinase family has been associated with the ageing of human cartilage cells (17). Consecutive studies showed that the determination of the enzymatic activity of chitobiosidases, chitinases, chitibiases and/or N-acetylglucosaminidases can be used for determining the age and the risk of developing age-related diseases and cancer in humans. In this case, all or some individual activities of chitobiosidases, chitinases, chitibiases and N-acetylglucosaminidases are measured.
3. Cathelicidin-related anti-microbial protein (CRAMP, in humans also referred to as LL-37): This protein is activated in response to activation of the innate immune system and seems to have a function in the protection against bacterial infections (14).
4. Stathmin (OP18): This protein controls the stability of microtubuli, cell motility and mitosis (20).

The determination is preferably effected from blood or serum samples.

It is found that these four protein markers are up-regulated in various organs of telomere-dysfunctional mice (kidney, liver, lung, brain, spleen and heart). In addition, the protein expression of these marker proteins is increased in the blood serum of ageing mice with dysfunctional telomeres. These markers appear to be specific for ageing due to telomere dysfunction since an up-regulation of these marker proteins does not occur in wild type mice with long telomeres. The studies also show that the same marker proteins are up-regulated in ageing human cells (fibroblasts) in the course of ageing and in response to radiation-induced DNA damage in young human cells.

Orthologous proteins of the marker proteins identified in the mouse system are known for three of the four proteins in humans: EF-1alpha, stathmin, CRAMP. An orthologue of Chi3L3 is not currently known in humans. However, it is possible to determine the enzyme activity of chitobiosidases, chitinases, chitibiases and N-acetylglucosaminidases in human samples. The studies showed for the first time that these enzyme activities can be used for determining the age and risk of developing age-related diseases and cancer. The determination of chitobiosidases is particularly preferred.

An essential feature of ageing is the accumulation of DNA damage. The accumulation of telomere dysfunction is also to be understood before this background, since an activation of DNA damage signal pathways occurs in cells in response to telomere dysfunction (21). A number of premature ageing syndromes in humans is related to the mutation of genes necessary for maintaining DNA stability. Our own studies have shown that the identified marker proteins are up-regulated also in human cells in response to radiation-induced DNA damage. Thus a significant up-regulation of the marker proteins on the RNA and protein levels occurs in response to irradiation. In addition, an up-regulation of the marker proteins can be detected in the cell culture medium of irradiated human cells as compared to non-irradiated human cells.

Further, methods for detecting the 3 orthologous marker proteins in the blood serum of humans by means of ELISA have been established. In addition, a commercially available kit for determining the enzyme activity of chitinases, chitibiases and N-acetylglucosaminidases in human samples has been provided.

Further processes for the detection of the marker proteins include quantitative PCRs for the marker proteins. Further, additional antibodies that can be used for the immunohistochemical detection of the marker proteins in human tissue samples have been defined.

The identified proteins are biomarkers for DNA damage and telomere dysfunction and can be used for determining the biological age, regenerative capacity, cancer risk, the risk of developing age-related diseases and for the prognosis in chronic diseases in humans and animals. The processes relate to ex vivo examinations of body fluids or biopsies.

The process can be applied to mammals and, in particular, humans. Ex vivo determination is preferred.

The following objects can be achieved by the process according to the invention:

1. Determination of the presence of DNA damage and dysfunctional telomeres

DNA damage and telomere dysfunction are fundamental mechanisms underlying the development of age-related diseases, ageing, declining regeneration capacity and cancer. The detection of DNA damage and telomere dysfunction is difficult. There are currently no readily detectable serum markers that can be determined in the blood of body fluids and indicate the presence of DNA breaks or telomere dysfunction. The defined markers can be used for this application. The examinations show for the first time that the identified markers increase in the blood serum in response to telomere dysfunction or DNA damage. Due to the increasing awareness that DNA damage and telomere dysfunction are fundamentally underlying the development of age-related diseases and cancer, the invention represents a substantial progress in medicine and provides new biomarkers that can be used.

2. Determination of biological age and life expectancy:

The biological age of an individual can deviate from their chronological age. It is known that genetic factors, living conditions, living habits, eating habits, external factors and many other factors have an influence on ageing in an organism. The biological age in part has a stronger influence on the life expectancy and fitness of the ageing individual than their chronological age. Slowly aged 60-year old humans can in part be fitter and have a longer life expectancy than prematurely aged 50 year old ones.

The measurement of the expression of the biomarkers as herein defined can determine the presence and the extent of DNA damage and telomere dysfunction. There is a growing body of evidence that these two parameters correlate with the biological age of an individual and their life expectancy. The measurement can be performed in body fluids (e.g., serum, blood, urine, saliva, cerebrospinal fluid) or in tissue and organ biopsies and samples. In addition to measurement by staining, PCR and gene array, the markers can also be determined by modern imaging methods (molecular imaging). These methods are suitable for determining the ageing condition of organs or for identifying aged cell clones with increased risk of degeneration.

The determination of the biological age and the life expectancy by means of these markers is suitable for the following fields:
a) Personal life plans: For individual life plans, it is important to be able to estimate how long one will presumably be fit in general and fit for work and what will be their own life expectancy. The determination of the biomarkers as herein defined can be used for determining the biological age and life expectancy of the individual. This can help the individual in his personal life plans.
b) Medical field: In the medical field, it is important to therapy planning to be able to determine the biological age and life expectancy of a patient. This is of importance, in particular, in invasive therapies (surgery, intensive care medicine, organ transplantation etc.). The determination of the biomarkers as herein defined can be used for determining the biological age and life expectancy of the individual. This can help physicians to better set individual therapy indications.
c) Lifestyle and wellness industry: There are indications that sports and wellness applications can have a beneficial influence on ageing. It is not known what programs/applications are best suitable for whom to delay ageing. In addition, it is not possible to objectively determine the success of such methods. The determination of the biomarkers as herein defined can be used to optimize the use of such methods and to evaluate the success of the measures in the course thereof.
d) Healthy foods/food additives: There are indications that the diet can have a beneficial influence on ageing. It is not known what foods/food additives are best suitable for whom to delay ageing. In addition, it is difficult to determine the success of such methods. The determination of the biomarkers as herein defined can be used to optimize the use of such methods and to evaluate the success of the measures in the course thereof.
e) Medicaments/substances for improving the fitness, organ function and life expectancy: There are a steadily increasing number of medicaments and substances that may have a beneficial influence on ageing and fitness in old age. It is not known what medicaments/substances are best suitable for whom to delay ageing. In addition, it is difficult to determine the success of such medicaments/substances. The determination of the biomarkers as herein defined can be used to optimize the use of such medicaments/substances and to evaluate the success of the measures in the course thereof.
f) Forensic science/criminalistics: The determination of the markers as herein defined can be used to determine the biological age of victims of violent crimes and accidents.
g) Determination of the biological age and life expectancy of farm animals, pets and animals employed in sports:
   The trade with farm animals, pets and animals employed in sports (e.g., racing horses, showjumpers, camels, hounds etc.) is often accompanied by medical expert opinions relating to the fitness and expected useful life of the animals. The measurement of the markers as herein defined can be used to determine the biological age and expected fitness span and life expectancy of animals.
h) Sports medicine/professional sports: The determination of the markers as mentioned above can be used to determine the biological age and fitness of athletes.
i) Insurances: The determination of the markers as mentioned above can be used to determine the biological age and fitness of insurants.
j) Occupational/environmental medicine: The determination of the markers as herein defined can be used to determine the influence of certain occupations and the influence of environmental factors on the biological ageing and fitness of individuals.
   3. There are experimental indications that the accumulation of DNA damage and telomere dysfunction limits the regeneration capacity of tissues and organs. Therefore, the biomarkers as herein defined can be used to determine the regeneration capacity of tissues and organs. The measurement can be made in body fluids (e.g., serum, blood, urine, saliva, cerebrospinal fluid) or in tissue and organ biopsies/samples. The determination of the regeneration capacity of organs and tissues by means of these markers is suitable for the following fields:
a) Determination of the prognosis and therapy planning in chronic diseases: A number of chronic diseases causes the afflicted organs to fail in the final stage. The courses can be very different between individuals. The prediction of the individual course is clinically important to be better able to plan the timing of invasive therapies (e.g., organ transplantations). The determination of the markers as herein defined can be used to determine the individual prognosis in chronic diseases (e.g., hepatitis, pulmonary fibrosis, anemias, chronic inflammatory diseases).
b) Determination of the prognosis and therapy planning in acute diseases and injuries.
   A number of acute diseases and injuries can cause the afflicted organs to fail and the patients to die. The courses can be very different between individuals. The prediction of the individual course is clinically important to estimate the timing and usefulness of invasive therapies (e.g., surgery, intensive care measures). The determination of the markers as herein defined can be used to determine the individual prognosis in acute diseases and injuries.
      4. There are increasing indications that the accumulation of DNA damage and telomere dysfunction determines the risk of occurrence and the prognosis of age-related diseases. Therefore, the determination of the markers as herein defined can be used to determine the risk of occurrence and the prognosis of age-related diseases. The measurement can be performed in body fluids (e.g., serum, blood, urine, saliva, cerebrospinal fluid) or in tissue and organ biopsies/samples.

The determination of the risk of occurrence and the prognosis of age-related diseases by means of the markers as herein defined is suitable for the following fields:
a) Determination of the risk of occurrence of age-related diseases: A wide variety of diseases is related to ageing (vascular diseases, diabetes mellitus, dementia, strokes etc.). It would be of clinical importance to be able to estimate the risk of occurrence of such diseases in order to begin early with preventive or therapeutic countermeasures, should the case arise. The determination of the markers as herein defined can be used to determine the individual risk to become afflicted with age-related diseases.
b) Determination of the prognosis of age-related diseases: A wide variety of diseases is related to ageing (vascular diseases, diabetes mellitus, dementia, strokes etc.). It would be of clinical importance to be able to estimate the prognosis of such diseases in order to take therapeutic measures adapted to the individual course. The determination of the markers as herein defined can be used to determine the individual course and prognosis in age-related diseases.
   5. There are increasing indications that DNA damage and telomere dysfunction results in the formation of cancer. Therefore, the determination of the markers as herein defined can be used to determine the risk of cancer in chronic diseases and in the course of ageing. The measurement can be performed in body fluids (e.g., serum, blood, urine, saliva, cerebrospinal fluid) or in tissue and organ biopsies/samples.

The determination of the risk of cancer by means of the markers as herein defined is suitable for the following fields:
a) Determination of the risk of cancer in the course of ageing. In the course of ageing, the risk for cancer increases. The markers as herein defined indicate the risk of cancer in the course of ageing. A determination of the risk of cancer can be used to adapt cancer screening to the individual risk for cancer.
b) Determination of the risk of cancer in chronic diseases. In many chronic diseases (e.g., hepatitis, inflammatory bowel diseases) the risk for cancer increases. The markers as herein defined indicate the individual risk of cancer in chronic diseases. A determination of the risk of cancer can be used to adapt cancer screening to the individual risk for cancer. In addition, the timing of preventive measures (surgery/transplantation) can be improved.
c) Determination of the risk of cancer in genetic predisposition and in genetic diseases. Many genetic diseases (e.g., Li-Fraumeni syndrome, adenomatosis polyposis coli) are accompanied by an increased risk for cancer. The determination of the markers as herein defined can indicate the individual risk of cancer in genetic predisposition and thus be used to improve cancer screening and for an improved timing of preventive measures in such diseases.
d) General cancer screening: The “general cancer screening” is recommended for many cancer types (e.g., colon carcinoma, prostate carcinoma, breast cancer) in the course of ageing and follows general medical guidelines for the time and frequency of the screening. The individual risk of becoming afflicted with cancer is not taken into account in these measures. The determination of the markers as herein defined can indicate the individual risk of cancer and can be used for an improved “general cancer screening” adapted to the individual risk.

### DESCRIPTION OF THE FIGURES

FIG. 1: The markers of telomere dysfunction and DNA damage are detectable in blood and indicate the risk of tumors in the course of ageing and in chronic liver disease.

A) The serum level of EF1alpha is significantly increased in the blood of patients infected with hepatitis C virus who developed liver cancer in the course of the disease (group 1) as compared to patients who did not develop liver cancer in the same observation period (group 2, p=0.02).

B) 85 year old subjects with an increased EF1alpha serum level (50% of the subjects above the mean serum level=blue line) showed a significantly higher risk of becoming afflicted with cancer in the course of 4 years as compared to 85 year old subjects with a lower EF1alpha serum level (50% of the subjects below the mean serum level=red line, p=0.002).

C) The chitinase enzyme activity is significantly increased in the blood of patients infected with hepatitis C virus who developed liver cancer in the course of the disease (group 1) as compared to patients who did not develop liver cancer in the same observation period (group 2, p=0.02).

D) 85 year old subjects with an increased CRAMP serum level (50% of the subjects above the mean serum level=blue line) showed a significantly higher risk of becoming afflicted with cancer in the course of 4 years as compared to 85 year old subjects with a lower CRAMP serum level (50% of the subjects below the mean serum level=red line, p=0.002).

FIG. 2: The markers of telomere dysfunction and DNA damage are detectable in blood and are influenced by lifestyle (smoking, sports, adiposity).

A-C) The protein levels of EF1alpha and stathmin and the chitinase enzyme activity in human blood serum exhibit a significant negative correlation with the level of exercise. These data indicate that a higher level of exercise is associated with reduced DNA damage.

D) The protein level of stathmin and the chitinase enzyme activity in human blood serum exhibit a significant positive correlation with cigarette smoking (measured in pack years=years of life in which one pack of cigarettes was smoked per day). These data indicate that smoking is associated with increased DNA damage.

E-G) The protein levels of EF1alpha, stathmin and CRAMP in human blood serum exhibit a significant positive correlation with adiposity (measured as body mass index. These data indicate that adiposity is associated with increased DNA damage.

The invention will be further illustrated by the following Examples.

### EXAMPLE 1

EF-1alpha: The up-regulation of this protein has been related to the proliferation loss (senescence) of human cells in culture (18, 19). A connection with human ageing and age-related diseases has not been described. Further, it has not been shown that EF1alpha is up-regulated by DNA damage and telomere dysfunction. Further, it has not been shown that EF1alpha is up-regulated by DNA damage and telomere dysfunction.

The studies show for the first time that this protein increases in the blood serum in response to telomere dysfunction and DNA damage (Jiang et al., 2009). In addition, the studies show for the first time that EF-1alpha protein is detectable in human blood and increases in the course of ageing and in age-related diseases and chronic diseases (Jiang et al., 2009). Thus, the blood serum level of EF-1alpha is significantly higher in elderly people in old people's homes (n=20, average age 85 years, EF-1alpha=1.5 units) as compared to young people (n=31, average age 35 years, EF-1alpha=1 unit, p=0.0004). Another increase can be observed with geriatric patients (n=72, average age 73 years, EF-1alpha=1.7 units, p=0.0115). In addition, the marker showed an increased expression in the final stage of chronic diseases (e.g., liver cirrhosis and myelodysplastic syndromes) both in the blood serum and in the afflicted tissues.

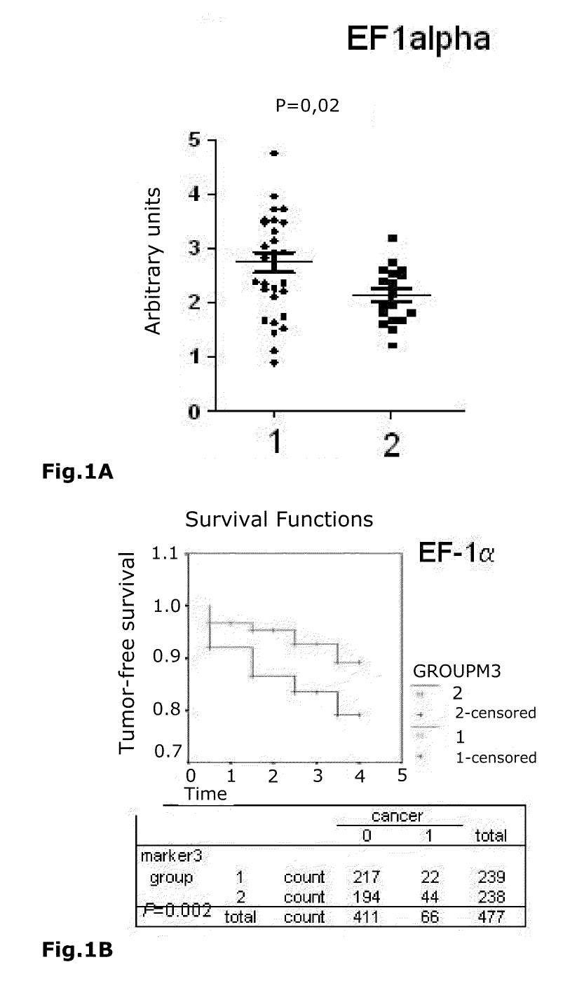

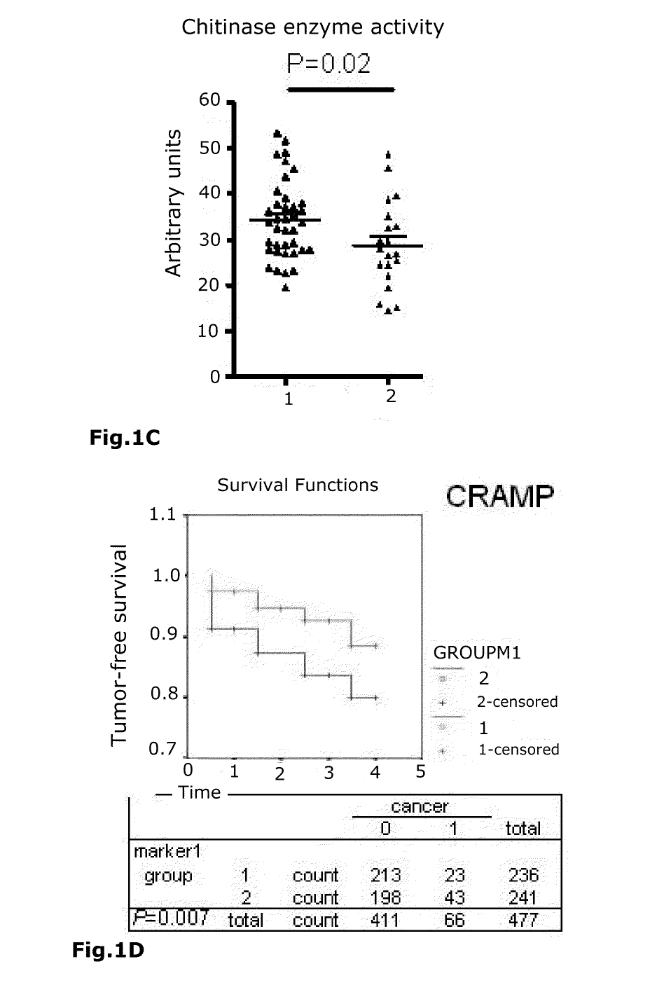

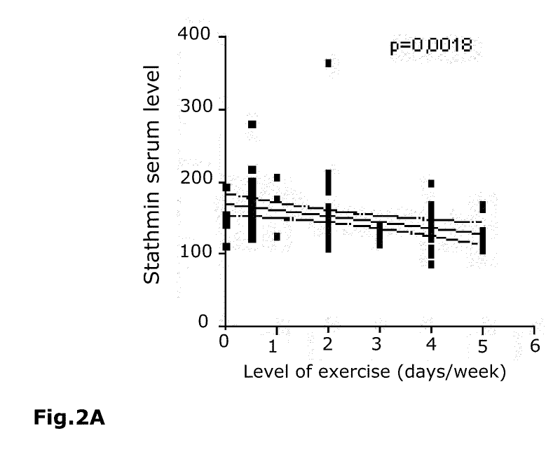

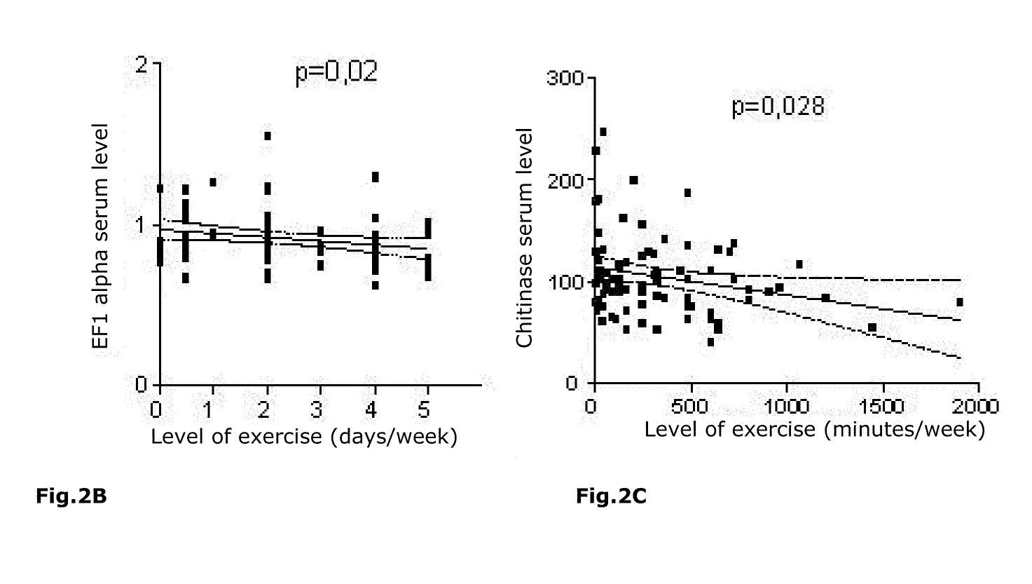

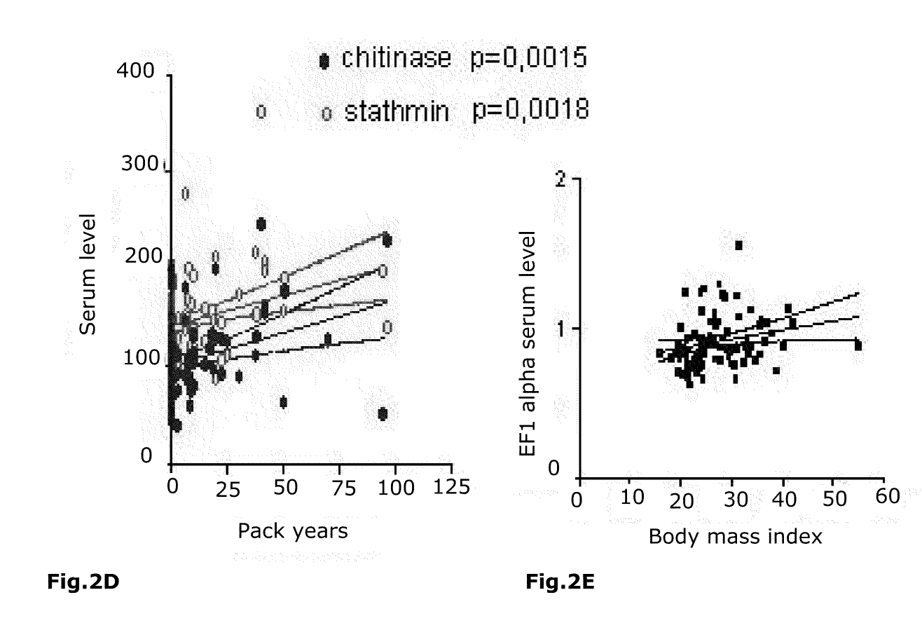

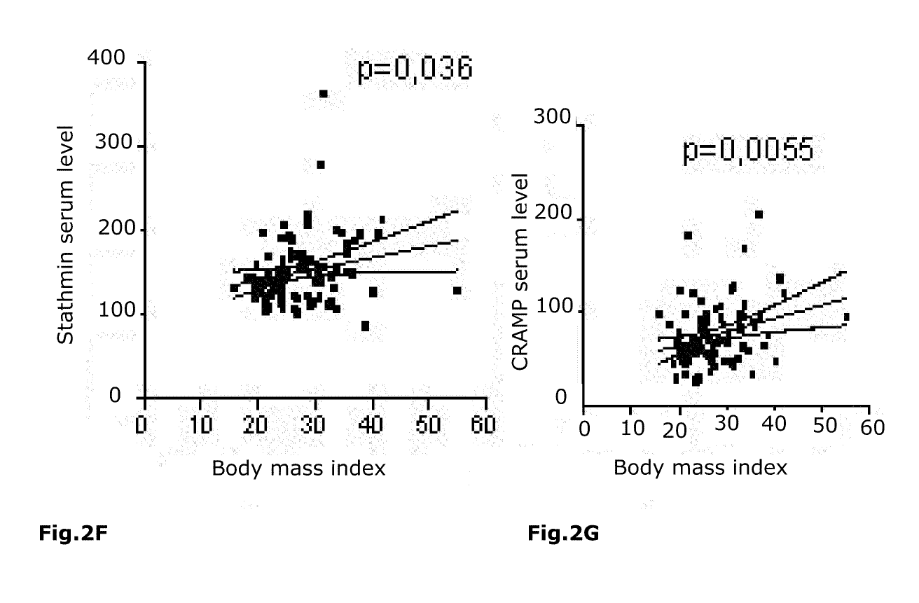

In addition, the studies show for the first time that the serum protein levels of the marker indicate the risk of cancer in old age and in chronic diseases (FIG. 1A). In >85 year old subjects, the risk of becoming afflicted with cancer within the next 4 years was significantly higher if the EF-1alpha serum level was increased (> median, 46/243 subjects developed tumors) as compared with the subjects having a lower EF-1alpha serum level (<median, 22/243 subjects developed tumors, p=0.001). In addition, the EF-1alpha serum level in liver cirrhosis patients who developed liver cancer in the course of the disease was significantly higher as compared to liver cirrhosis patients who did not develop liver cancer (FIG. 1B).

In addition, our studies show for the first time that the serum protein levels of the marker are significantly influenced by lifestyle (sports and adiposity, FIGS. 2A, E).

### EXAMPLE 2

CRAMP (also referred to as LL-37 in humans): The studies show for the first time that this protein rises in the blood serum in response to telomere dysfunction (22). In addition, our studies demonstrate for the first time that this protein increases in human blood in the course of human ageing and in the course of age-related diseases (22). Thus, the blood serum level of CRAMP is significantly higher in elderly people in old people's homes (n=20, average age 85 years, CRAMP=18 ng) as compared to young people (n=31, average age 35 years, CRAMP=8 ng, p<0.0001). Another increase can be observed with geriatric patients (n=72, average age 73 years, CRAMP=22 ng, p=0.0007). In addition, the marker shows an increased expression in the final stage of chronic diseases (e.g., liver cirrhosis and myelodysplastic syndromes) both in the blood serum and in the afflicted tissues. In addition, the marker indicates the risk of cancer in old age and in chronic diseases.

These studies show for the first time that the serum level of CRAMP (LL-37) in human blood in 85 year old subjects indicates the risk of becoming afflicted with cancer within the next 4 years (FIG. 1C). Subjects having an increased CRAMP serum level (> median) showed significantly higher rates of development of malignant tumors (44/243 subjects developed tumors) as compared with the subjects having a lower CRAMP serum level (<median, 24/243 subjects developed tumors, p=0.006). In addition, the CRAMP serum level in liver cirrhosis patients with liver cancer was significantly higher as compared to liver cirrhosis patients without liver cancer.

In addition, the studies show for the first time that the serum level of CRAMP is significantly influenced by adiposity (FIG. 2G).

### EXAMPLE 3

Stathmin: The studies show for the first time that this protein rises in the blood serum in response to telomere dysfunction (22). In addition, the studies demonstrate for the first time that this protein increases in human blood in the course of human ageing and in the course of age-related diseases (22). Thus, the blood serum level of stathmin is significantly higher in elderly people in old people's homes (n=20, average age 85 years, stathmin=1.3 units) as compared to young people (n=31, average age 35 years, stathmin=1 unit, p=0.0001). In addition, the marker showed an increased expression in the final stage of chronic diseases (e.g., liver cirrhosis and myelodysplastic syndromes) both in the blood serum and in the afflicted tissues.

In addition, the marker indicates the risk of cancer in old age and in chronic diseases. The stathmin serum level in liver cirrhosis patients with liver cancer is significantly higher as compared to liver cirrhosis patients without liver cancer.

In addition, the studies show for the first time that the serum protein levels of the marker are significantly influenced by lifestyle (sports, smoking and adiposity (FIGS. 2A, D, F).

### EXAMPLE 4

Enzyme activity of chitinases, chitibiases and N-acetylglucosaminidases: An increase of the secretion of chitinase-like protein in the cell culture of cartilage cells of aged humans and in arthritis patients has been described (17). An increase of the enzyme activity of chitinases, chitibiases and N-acetylglucosaminidases in human blood or in human tissues/organs as a consequence of DNA damage, telomere dysfunction, ageing or diseases has not yet been described.

The studies show for the first time that the enzyme activities of chitobiosidases, chitinases, chitibiases and N-acetylglucosaminidases in blood serum increase in response to telomere dysfunction (22). In addition, the studies demonstrate for the first time that these enzyme activities increase in human blood in the course of human ageing and in age-related diseases (22). Thus, the enzyme activity of chitinases, chitibiases and N-acetylglucosaminidases is significantly higher in the blood serum of elderly people in old people's homes (n=20, average age 85 years, chitinase enzyme activity 52 units) as compared to young people (n=31, average age 35 years, enzyme activity=24 units, p=0.0004). Another increase can be observed with geriatric patients (n=72, average age 73 years, enzyme activity=56 units, p=0.0216). In addition, the enzyme activity of chitinases, chitibiases and N-acetylglucosaminidases is increased in the final stage of chronic diseases (e.g., liver cirrhosis and myelodysplastic syndromes) in the blood serum.

In addition, the studies show for the first time that the enzyme activity of chitobiosidases, chitinases, chitibiases and N-acetylglucosaminidases measured in blood serum indicate the risk of cancer in chronic diseases. The enzyme activity of chitobiosidases, chitinases, chitibiases and N-acetylglucosaminidases in liver cirrhosis patients with liver cancer was significantly higher as compared to liver cirrhosis patients who did not develop liver cancer in the same follow-up period (FIG. 1D).

In addition, the studies show for the first time that the chitinase enzyme activity is significantly influenced by lifestyle (sports and smoking, FIGS. 2CA, D).

### REFERENCES

1. Wolfgang Lutz, Warren Sanderson & Sergei Scherbov. The coming acceleration of global population ageing. Nature 451, 716-719
2. Chan S R, Blackburn E H. Telomeres and telomerase. Philos Trans R Soc Lond B Biol Sci. 2004; 359:109-21.
3. Allsopp R C, Vaziri H, Patterson C, Goldstein S, Younglai E V, Futcher A B, Greider C W, Harley C B. Telomere length predicts replicative capacity of human fibroblasts. Proc Natl Acad Sci USA. 1992 Nov. 1; 89(21):10114-8.
4. Jiang H, Ju Z, Rudolph K L. Telomere shortening and ageing. Z Gerontol Geriatry 2007; 40:314-324.
5. Cawthon R M, Smith K R, O'Brien E, Sivatchenko A, Kerber R A. Association between telomere length in blood and mortality in people aged 60 years or older. Lancet. 2003 Feb. 1; 361(9355):393-5.
6. Panossian L A, Porter V R, Valenzuela H F, Zhu X, Reback E, Masterman D, Cummings J L, Effros R B. Telomere shortening in T cells correlates with Alzheimer's disease status. Neurobiol Aging. 2003; 24:77-84.
7. Jeanclos E, Krolewski A, Skurnick J, Kimura M, Aviv H, Warram J H, Aviv A. Shortened telomere length in white blood cells of patients with IDDM. Diabetes. 1998; 47:482-6.
8. Minamino T, Komuro I. Vascular cell senescence: contribution to atherosclerosis. Circ Res. 2007 Jan. 5; 100(1):15-26.
9. Wu X, Amos C I, Zhu Y, Zhao H, Grossman B H, Shay J W, Luo S, Hong W K, Spitz M R. Telomere dysfunction: a potential cancer predisposition factor. J Natl Cancer Inst. 2003 Aug. 20; 95(16):1211-8.
10. Wiemann S U, Satyanarayana A, Tsahuridu M, Tillmann H L, Zender L, Klempnauer J, Flemming P, Franco S, Blasco M A, Manns M P, Rudolph K L. Hepatocyte telomere shortening and senescence are general markers of human liver cirrhosis. FASEB J. 2002; 16:935-42.
11. Ohyashiki J H, Ohyashiki K, Fujimura T, Kawakubo K, Shimamoto T, Iwabuchi A, Toyama K. Telomere shortening associated with disease evolution patterns in myelodysplastic syndromes. Cancer Res. 1994 Jul. 1; 54(13): 3557-60.
12. Hemann M T, Strong M A, Hao L Y, Greider C W. The shortest telomere, not average telomere length, is critical for cell viability and chromosome stability. Cell. 2001 Oct. 5; 107(1):67-77.
13. Zou Y, Sfeir A, Gryaznov S M, Shay J W, Wright W E. Does a sentinel or a subset of short telomeres determine replicative senescence? Mol Biol Cell. 2004 August; 15(8): 3709-18.
14. Nizet V, Ohtake T, Lauth X, Trowbridge J, Rudisill J, et al. (2001). Innate antimicrobial peptide protects the skin from invasive bacterial infection. Nature 414: 454-457.
15. Zhu Z, Zheng T, Horner R J, Kim Y K, Chen Ny, et al. (2004). Acidic Mammalian Chitinase in Asthmatic Th2 Inflammation and IL-13 Pathway Activation. Science 304:1678-1682.
16. Reese T A, Liang H E, Tager A M, Luster A D, Van Rooijen N, et al. (2007). Chitin induces accumulation in tissue of innate immune cells associated with allergy. Nature 447: 92-96.
17. Dozin B, Malpeli M, Camardella L, Cancedda R, Pietrangelo A. (2002). Response of young, aged and osteoarthritic human articular chondrocytes to inflammatory cytokines: molecular and cellular aspects. Matrix Biol 21: 449-459.
18. Wang E, Moutsatsos I K, & Nakamura T. (1989). Cloning and molecular characterization of a cDNA clone to statin, a protein specifically expressed in nonproliferating quiescent and senescent fibroblasts. Exp Gerontol 24: 485-49
19. Giordano T, Kleinsek D, & Foster D N. (1989). Increase in abundance of a transcript hybridizing to elongation factor 1 alpha during cellular senescence and quiescence. Exp Gerontol 24: 501-513.
20. Rubin C I, & Atweh G F. (2004). The role of stathmin in the regulation of the cell cycle. J Cell Biochem 93: 242-250.
21. d'Adda di Fagagna et al. (2003). A DNA damage checkpoint response in telomere-initiated senescence. Nature 426:194-8.
22. Jiang H, Schiffer E, Song Z, Wang J, Zürbig P, Thedieck K, Moes S, Saal N, Bantel H, Jantos J, Brecht M, Jenö P, Hall M N, Hager K, Manns M P, Hecker H, Ganser A, Döhner K, Bartke A, Meissner C, Mischak H, Ju Z, Rudolph K L. Proteins induced by telomere dysfunction and DNA damage represent biomarkers of human aging and disease. Proc Nat Acad Sci 2008, 105:11299-304

1. A process for determining the presence and extent of DNA damage and telomere dysfunction in humans or animals, comprising the following steps:
determining the level or activity of at least one protein marker in a blood or serum sample, said protein being selected from the group consisting of EF1α, chitobiosidases, stathmin and CRAMP.

2. A process for determining the ageing condition of an organism, comprising the following steps:
determining the level or activity of at least one protein marker in a sample, said protein being selected from the group consisting of (i) EF1α, (ii) CRAMP, (iii) stathmin and (iv) chitinase gene family.

3. The process according to claim 1, wherein the enzyme activity of chitobiosidases, chitinases, chitibiases and/or N-acetylglucosaminidases is measured as said activity of the chitinase gene family.

4. The process according to claim 1, wherein the measurement of the expression is effected by:
measuring the protein;
measuring fragments of the protein;
measuring mRNA coding for the protein;
measuring mRNA fragments coding for the protein;
measuring the biological activity of the protein;
pictorial representation of the proteins or the activity of the proteins (molecular imaging).

5. The process according to claim 1, wherein the measurement is performed in body fluids or in tissue and organ biopsies or samples.

6. The process according to claim 5, wherein said body fluids are selected from blood, urine, saliva and cerebrospinal fluid, especially serum.

7. The process according to claim 2, wherein said process serves for determining the biological age, the life expectancy, the regenerative capacity of tissues and organs, the risk of occurrence and the prognosis of age-related diseases, for determining the risk of cancer in chronic diseases, for determining the risk of cancer in the course of ageing, for determining the risk of cancer in the course of chronic diseases.

8. The process according to claim 1, wherein 2, 3 or 4 protein markers from the group of (i) CRAMP, (ii) EF1α, (iii) stathmin and (iv) chitinase gene family are determined.

9. The process according to claim 8, wherein the activity of chitinases is determined as the enzyme activity of chitobiosidases, chitinases, chitibiases and/or N-acetylglucosaminidases.

10. The process according to claim 1, wherein the markers or the marker activity are represented by imaging (molecular imaging).

11. The process according to claim 2, wherein the enzyme activity of chitobiosidases, chitinases, chitibiases and/or N-acetylglucosaminidases is measured as said activity of the chitinase gene family.

12. The process according to claim 2, wherein the measurement of the expression is effected by:
measuring the protein;
measuring fragments of the protein;
measuring mRNA coding for the protein;
measuring mRNA fragments coding for the protein;
measuring the biological activity of the protein;
pictorial representation of the proteins or the activity of the proteins (molecular imaging).

13. The process according to claim 2, wherein the measurement is performed in body fluids or in tissue and organ biopsies or samples.

14. The process according to claim 2, wherein said body fluids are selected from blood, urine, saliva and cerebrospinal fluid, especially serum.

15. The process according to claim 2, wherein 2, 3 or 4 protein markers from the group of (i) CRAMP, (ii) EF1α, (iii) stathmin and (iv) chitinase gene family are determined.

16. The process according to claim 2, wherein the activity of chitinases is determined as the enzyme activity of chitobiosidases, chitinases, chitibiases and/or N-acetylglucosaminidases.

17. The process according to claim 2, wherein the markers or the marker activity are represented by imaging (molecular imaging).
